(19) United States
(12) Patent Application Publication
(10) Pub. No.: US 2007/0123739 A1
(43) Pub. Date: May 31, 2007

(54) Hydrogenolysis of sugar feedstock

(75) Inventors: Simon Peter Crabtree, London (GB); Derek Vincent Tyers, London (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Davy Process Technology Limited, London, England (GB)

(21) Appl. No.: 10/576,487

(22) PCT Filed: Oct. 15, 2004
(86) PCT No.: PCT/GB04/04391

§ 371 Date: Apr. 21, 2006

(30) Foreign Application Priority Data
Oct. 30, 2003 (GB) 0325385.1

(51) Int. Cl. C07C 31/18; C07C 29/14

(52) U.S. Cl. 568/863

(57) ABSTRACT

A process for the hydrogenolysis of a sugar feedstock in the presence of a catalyst comprising: (a) ruthenium or osmium; and (b) an organic phosphine; and wherein the hydrogenolysis is carried out in the presence of water and at a temperature of greater than 150° C.

The present invention relates to a homogeneous process for the production of glycols from sugar derived feedstocks. More particularly, it relates to a homogeneous hydrogenolysis process which can be carried out in the presence of water. Most particularly it relates to a homogeneous hydrogenolysis process for a feedstock comprising one or more of polyols, alditols, aldoses, polymers of aldoses and starch.

For ease of reference the feedstock comprising one or more of polyols, alditols, aldoses, polymers of aldoses such as starch and cellulose will be described generally as a “sugar feedstock”. The polymers of aldoses include homopolymers and copolymers.

Many catalyst systems are known which are suitable for use in the hydrogenolysis of sugars. Traditionally such reactions are carried out using heterogenous catalysts and often high temperature and pressures. Typically temperatures in the range of about 200° C. to about 275° C. are required with pressures in the region of from about 1000 psig to about 4000 psig. Many of these require the use of basic promoters to prevent catalyst degradation and/or to promote catalyst activity. However, the use of these promotors adds significantly to the cost of the reaction. The use of sulphur containing additives have been suggested to increase the selectivity of the catalyst. However, this increase in selectivity is often at the expense of a loss of activity. Examples of heterogeneous processes can be found in U.S. Pat. No. 6,479,713, U.S. Pat. No. 6,291,725, U.S. Pat. No. 5,326,912, U.S. Pat. No. 5,354,914, U.S. Pat. No. 5,600,028, U.S. Pat. No. 5,403,805, U.S. Pat. No. 5,210,335, U.S. Pat. No. 5,107,018, U.S. Pat. No. 5,107,018, FR2603276, U.S. Pat. No. 4,496,780, U.S. Pat. No. 4,476,331, U.S. Pat. No. 443,184, U.S. Pat. No. 4,401,823, U.S. Pat. No. 4,380,678, U.S. Pat. No. 4,404,411, U.S. Pat. No. 4,366,332, GB988040, U.S. Pat. No. 3,011,002, U.S. Pat. No. 282,603, GB490211, GB430576, Abreau et al, Biomass and Bioenergy 9, 587 (1995) and J.Catalysis 208 248 (2002) Fabre et al.

Homogeneous process have also been suggested and examples of these can be found in U.S. Pat. No. 5,118,883, U.S. Pat. No. 5,026,927, U.S. Pat. No. 3,935,284, U.S. Pat. No. 6,080,898, U.S. Pat. No. 4,642,394, U.S. Pat. No. 5,097,089, U.S. Pat. No. 3,454,644, J.Organomet. Chem. 417 41 (1991) G Braca et al, J. Molecular Catal. 22 138 (1983) and J. Molecular Catal. 16 349 (1982).

Whilst some of these processes go some way to providing a commercial process, they suffer from certain disadvantages and drawbacks. In particular, they are costly to operate, many require the presence of a strong basic promoter and are temperature sensitive. For example, the process of U.S. Pat. No. 5,026,927 operates at a temperature of from 75° C. to about 150° C. and that of U.S. Pat. No. 3,935,284 requires a temperature of 150° C. or less. It is stated in U.S. Pat. No. 3,935,284 that at temperatures in excess of 150° C., decarbonylation occurs to produce a carbonyl-ruthenium species which is a less active catalyst.

It is therefore desirable to provide a process which provides a cost-effective process for sugar hydrogenolysis and which utilises a catalyst regime that has the required levels of selectivity and activity.

Thus according to the present invention there is provided a process for the hydrogenolysis of a sugar feedstock in the presence of a catalyst comprising:
(a) ruthenium or osmium; and
(b) an organic phosphine;

and wherein the hydrogenolysis is carried out in the presence of water and at a temperature of greater than 150° C.

By “homogeneous process” we mean that the catalyst is dissolved in the solvent for the reaction and that at least some of the water present and at least some of the sugar feedstock must be in phase with the catalyst. Where excess water and/or excess feedstock is present, the excess may form a separate phase to that comprising the catalyst. Additionally, or alternatively, the product may form a separate phase.

As detailed above, the sugar feedstock may be a feedstock comprising one or more of polyols, alditols, aldoses and polymers of aldoses such as cellulose and starch. Examples of alditols and aldoses suitable for use in the process of the present invention include those having from C₃ to C₁₂, more particularly C₃ to C₆. Examples of suitable feedstocks include glucose, sucrose, xylose, arabinose, mannose, mannitol, sorbitol, xylitol, arabinol, glycerol and mixtures thereof. The sugar feedstock may be provided from natural or synthetic sources or mixtures thereof.

Where the sugar feedstock is water soluble, the water may be present as the solvent for the reaction. Alternatively, a solvent may be used. Where a solvent is used, the water will be present as an additive in the solvent. In another alternative arrangement, the sugar feedstock or the product of the reaction may be the solvent. In one arrangement at least 1% by weight of water is present.

Where the sugar feedstock is non-water soluble or has low water-solubility, such as for example a sugar having a higher carbon content such as high molecular weight polymeric alditols, the feedstock or product may be the solvent for the reaction or an organic solvent may be used and the water may be present as an additive. In this case, it may be present in the solvent in any suitable amount and preferably in an amount of from about 1% up to the solubilitv limit of the water in the solvent. Additional water may be present in a separate aqueous phase.

The process of the present invention provides a method for the hydrogenolysis of sugars which can be carried out at higher temperatures than has been achievable heretofore to increase activity while maintaining the desired level of selectivity.

Further, it has been found that the presence of water is beneficial in terms of catalyst stability. It is noted that in prior art systems, decarbonylation is noted and the carbon monoxide formed is said to strongly inhibit the catalyst. Without wishing to be bound by any theory, it is believed that the presence of water allows a side reaction to occur in the hydrogenation reactor in which any carbon monoxide produced reacts with the water to form carbon dioxide and hydrogen via the water gas shift reaction. This carbon dioxide and hydrogen may be further reacted to form methane. These gases can be readily removed from the reaction system. It will therefore be appreciated that the need to provide a separate methanation unit in the recycling system for vent gases is obviated.

A further advantage of the present invention is that the removal of the carbon monoxide as detailed above allows for effective regeneration of the catalyst. Thus the process offers extended catalyst life which in turn improves the economics of the reaction.

As detailed above, where the sugar feedstock is soluble in water, the water may act as the solvent. However, the method of the present invention may be conducted in the absence of a solvent, i.e. the starting material or reaction product maybe a solvent for the reaction. However, if a solvent is used, any suitable solvent may be selected and examples of suitable solvents include, but are not limited to tetrahydrofuran, tetraethyleneglycol dimethyl ether, N-methyl pyrrolidone, diethyl ether, ethyleneglycol dimethylether, dioxane, 2-propanol, 2-butanol, secondary alcohols, tertiary alcohols, lactams and N-methyl caprolactam.

The catalyst of the present invention is a ruthenium/phosphine or osmium/phosphine catalyst with a ruthenium/phosphine catalyst being particularly preferred. The ruthenium is generally provided as a ruthenium compound although halides are not preferred. Suitable compounds are those which can be converted to active species under the reaction conditions and include nitrates, sulphates, carboxylates, beta diketones, and carbonyls. Ruthenium oxide, carbonyl ruthenates and complex compounds of ruthenium, including hydridophosphineruthenium complexes, may also be used. Specific examples include, but are not limited to, ruthenium nitrate, ruthenium dioxide, ruthenium tetraoxide, ruthenium dihydroxide, ruthenium acetylacetonate, ruthenium acetate, ruthenium maleate, ruthenium succinate, tris-(acetylacetone)ruthenium, pentacarbonylruthenium, dipotassium tetracarbonyl-ruthenium, cyclo-pentadienyldicarbonyltriruthenium, ruthenium dihydroxide, bis(tri-n-butylphosphine)tricarbonylruthenium, dodecacarbonyltriruthenium, tetrahydride-decacarbonyltetraruthenium, and undecacarbonylhydridetriruthenate. Corresponding compounds may be used where the catalyst is formed from osmium.

The catalyst maybe preformed or generated in situ. Where an electron rich phosphine such as tris-1,1,1-(diethyphosphinomethyl)ethane, is to be used it may be preferable to preform the catalyst in the absence of water prior to commencing the process of the present invention.

The ruthenium/osmium compound may be present in any suitable amount. However, it is preferably present in an amount of from 0.0001 to 5 mol, preferably 0.005 to 1 mol, as ruthenium/osmium per liter of reaction solution.

Any suitable phosphine may be used. Compounds which provide tridentate, bidentate and monodentate ligands may be used. Where the metal is ruthenium, tridentate phosphines are particularly preferred. Examples of suitable phosphine compounds include trialkylphosphines, dialkylphosphines, monoalkylphosphines, triarylphosphines, diarylphosphines, monoarylphosphines, diaryhuonoalkyl phosphines and dialkylmonoaryl phosphines. Specific examples include but are not limited to tris-1,1,1-(diphenylphosphinomethyl)methane, tris-1,1,1-(diphenylphosphinomethyl)-ethane, tris-1,1,1-(diphenylphosphinomethyl)propane, tris-1,1,1-(diphenylphosphino-methyl)butane, tris-1,1,1-(diphenylphosphinomethyl)-2,2dimethylpropane, tris-1,3,5-(diphenylphosphino-methyl)cyclohexane, tris-1,1,1-(dicyclo-hexylphosphinomethyl)ethane, tris-1,1,1-(dimethylphosphinomethyl)ethane, tris-1,1,1-(diethylphosphinomethyl)ethane, 1,5,9-triethyl-1,5-9-triphosphacyclododecane, 1,5,9-triphenyl-1,5-9-triphosphacyclododecane, bis(2-diphylephosphinoethyl)phenylphosphine, bis-1,2-(diphenyl phosphino)ethane, bis-1,3-(diphenyl phosphino)propane, bis-1,4-(diphenyl phosphino)butane, bis-1,2-(dimethyl phosphino)ethane, bis-1,3-(diethyl phosphino)propane, bis-1,4(dicyclohexyl phosphino)butane, tricyclohexylphosphine, trioctyl phosphine, trimethyl phosphine, tripyridyl phosphine, triphenylphosphine with tris-1,1,1-(diphenylphosphinomethyl)-ethane being particularly preferred. Particularly advantageous results are acheived with tridentate facially capped phosphines with tris-1,1,1-(diarylphosphinomethyl)alkane and tris-1,1,1-(diallcylphosphinomethyl)alkane being particularly preferred.

The phosphine compound may be present in any suitable amount. However, it is preferably present in an amount of from 0.0001 to 5 mol, preferably 0.005 to 1 mol, as phosphine per liter of reaction solution.

Whilst a strong base, such as potassium hydroxide, may be added they are not believed to have any significant benefit to the selectivity of the process. Examples of base additives include any of those identified in the prior art.

However, in one arrangement of the present invention an increase in selectivity may be noted where a second phosphine is present. The second phosphine will generally be a phosphine which is a more weakly coordinating ligand to the ruthenium or osmium than the first phosphine compound Examples of suitable second phosphines include triphenylphosphine and phosphine oxides such as triphenylphosphine oxide. Without wishing to be bound by any theory, these weakly co-ordinating ligands may compete with the active site at the metal thus preventing coordination of the product and thereby any undesirable side reaction from occurring. Alternatively, other weakly coordinating ligands such as amines may be used.

Any suitable reaction temperature in excess of 150° C. may be used. However, in the process of the present invention, particular advantages may be noted if the hydrogenolysis is carried out at temperatures in the region of from about 190° C. to about 260° C., more preferably 200° C. to about 250° C.

Any suitable pressure may be used with a reaction pressure of from about 250 psig to about 2000 psig, being preferred. More preferably a pressure of from 800 psig to 1200 psig may be used and most preferably a pressure of about 1000 psig may be used. However, it will be understood that if a volatile solvent is used a higher reactor pressure may be desirable due to the high partial pressure of the solvent in the reactor.

The process may be carried out either in a batch system or in a continuous system. High intensity reactors such as intensive gas/liquid mixing reactors may be used. However, it will be understood that the process of the present invention is particularly suitable for use in a continuous system since the catalyst is not poisoned by carbon monoxide or if poisoning in this way occurs, the catalyst can be regenerated by reaction with the water.

Where the catalyst is removed from the reactor, for example, with a product removal stream, it may be recycled by any suitable means to the reactor. The catalyst may be separated from the product stream by any suitable means. Suitable means include extraction, distillation, gas stripping and membrane separation. In some circumstances, the catalyst may be immobilised on a support to assist the recovery. In this arrangement, the immobilised catalyst may be recovered by filtration.

A pre-reduction step may be included to improve the selectivity to the desired product. In one arrangement, the pre-reduction step may be carried out in the same rector to the main reaction. In one alternative arrangement the pre-reduction may be carried out in a different reactor. Where the same reactor is used, the pre-reduction step may be carried out within different zones within the reactor or the same zone. Where the same reactor is to be used, different zones will generally be used for a continuous process. The pre-reduction step may be carried out at any suitable reaction conditions. However, generally it will be carried out at a lower temperature than that used for the main reaction. The temperature of the pre-reduction step may be from about 150° C. to about 250° C. and the pressure may be from about 600 to about 1000 psig. The pre-reduction step is found to be particularly useful where the sugar feedstock is an aldose. Whilst not wishing to be bound by any theory it is believed that the terminal aldehyde group of the aldose is reduced and that where the aldose is cyclic, the ring is opened. Some C—C bond cleavage may also occur.

The present invention will now be described with reference to the following examples which are not intended to be limiting on the scope of the invention.

### EXAMPLES 1 TO 5

These examples demonstrate the effect of varying the reaction temperature in a batch reaction.

0.18 g of ruthenium acetylacetonate (from Johnson Matthey), 0.38 g of 1,1,1(diphenylphosphino methyl)ethane) (from Aldrich) and tetrahydrofuran (from Aldrich), 20 g sorbitol (from Aldrich) and 50 g deionised water were weighed into a 300 ml Parr Hastelloy C autoclave which was then sealed. The headspace of the autoclave was purged before being pressurised to approximately 600 psig with hydrogen gas. The stirrer speed was 600 rpm and the reactor heated to the desired temperature. When the temperature was reached, the pressure in the reactor was increased to 1000 psig and the reaction time of 6 hours was considered to have started. The pressure in the autoclave was maintained throughout the reaction by feeding hydrogen gas under regulator control. At the end of the reaction the gas make up was stopped, and the reactor was cooled to room temperature before, the headspace was vented. The liquid products were removed and analysed on a Hewlett Packard HP6890 GC using a J&W 0.32 mm, 50 m, DB1, with a 1 μm phase thickness and using butoxyethanol as an internal standard for quantifying the amounts of propylene glycol, ethylene glycol and glycerol produced.

For the purpose of the results reported below, molar yield is considered to be 100 moles product/moles of feed. Hence if ethylene glycol were the only product a molar yield of 300% could, theoretically be reported for the conversion of sorbitol to products. For polymeric sugars, e.g. starch and sucrose they are considered to have the molecular weight of their monomer units for the molar yield calculation.

The results for various reaction temperatures are set out in Table 1

TABLE 1

| | | Ethylene | Propylene | | |
|---|---|---|---|---|---|
| Ex | Temp | glycol | glycol | glycerol | Total (Propylene glycol + |
| No | ° C. | (mol %) | (mol %) | (mol %) | Ethylene glycol) (mol %) |
| 1 | 250 | 48 | 82 | 8 | 130 |
| 2 | 250 | 50 | 80 | 2 | 130 |
| 3 | 225 | 51 | 68 | 50 | 119 |
| 4 | 200 | 57 | 62 | 41 | 119 |
| 5 | 190 | 42 | 46 | 46 | 88 |

### EXAMPLES 6 AND 7

These examples demonstrate the effect of pressure using a highly volatile solvent.

The method of Examples 1 to 5 was repeated at a temperature of 250° C. except that the pressure in the reactor was modulated. The results, which are set out in Table 2, indicate a dramatic loss in selectivity as the pressure is reduced.

TABLE 2

| | Pres- | Ethylene | Propylene | | Total (propylene |
|---|---|---|---|---|---|
| Ex | sure | glycol | glycol | Glycerol | glycol + ethylene |
| No | (psig) | (mol %) | (mol %) | (mol %) | glycol) (mol %) |
| 6 | 1000 | 48 | 82 | 8 | 130 |
| 7 | 750 | 27 | 27 | 5 | 54 |

### EXAMPLE 8 TO 13

This demonstrates that a range of solvents can be employed.

The method of Example 1 was repeated except that the solvent, tetrahydrofuran, was replaced with other solvents in varying amounts.

The results, which are set out in Table 3, illustrate that a range of solvents may be used.

TABLE 3

| | | Solvent | Ethylene | Propylene | | Total (Propylene |
|---|---|---|---|---|---|---|
| Ex | | Amount | glycol | glycol | Glycerol | glycol + Ethylene |
| No | Solvent | (g) | (mol %) | (mol %) | (mol %) | glycol) (mol %) |
| 8 | THF | 17.1 | 48 | 82 | 8 | 130 |
| 9 | iPA | 19.9 | 34 | 92 | 9 | 126 |
| 10 | TEGDE | 19.0 | 29 | 41 | <1 | 70 |
| 11 | TEGDE | 50 | 56 | 60 | 13 | 116 |
| 12 | NMP | 20.1 | 7 | 5 | 2 | 12 |
| 13 | NMP + | 74.8 | 104 | 59 | 1 | 163 |
| | THF | | | | | |

where THF = tetrahydrofuran, iPA = isopropanol; TEGDE = tetraethyleneglycol dimethylether and NMP = N-methyl pyrrolidone

### EXAMPLES 14 TO 18

These examples further demonstrate that a range of solvents may be employed and that their concentration may affect the observed selectivity.

The method of Example 1 was repeated except that the sorbitol was replaced with glucose and the quantity and nature of the solvent and amount of water present were varied.

The results are set out in Table 4.

TABLE 4

| | | Solvent | Water | Ethylene | Propylene | | Total (Propylene |
|---|---|---|---|---|---|---|---|
| Ex | | Amount | Amount | glycol | glycol | Glycerol | glycol + Ethylene |
| No | Solvent | (g) | (g) | (mol %) | (mol %) | (mol %) | glycol) (mol %) |
| 14 | THF | 20.0 | 50 | 30 | 91 | 5 | 121 |
| 15 | THF | 50.0 | 50 | 25 | 55 | 1 | 80 |
| 16 | NMP | 20.4 | 50 | 20 | 54 | 14 | 74 |
| 17 | NMP | 49.6 | 50 | 19 | 51 | 1 | 70 |
| 18 | NMP | 75.0 | 30 | 14 | 34 | 1 | 48 |

### EXAMPLES 19 TO 24

These examples demonstrate that the catalyst is suitable for the hydrogenation of a range of sugars as defined in the present invention.

The method of Example 1 was repeated except that the sorbitol was replaced by an alternative substrate.

The results are set out in Table 5. It is postulated that for the given conditions the sorbitol produced a higher yield than the cyclic sugars. Without wishing to be bound by any theory, it is believed that this is due to undesirable reactions occurring while the sugar is in the cyclised state.

TABLE 5

| | | | | | Total |
|---|---|---|---|---|---|
| | | | | | (Propylene |
| | | Ethylene | Propylene | | glycol + |
| Ex | | glycol | glycol | Glycerol | Ethylene |
| No | Substrate | (mol %) | (mol %) | (mol %) | glycol) (mol %) |
| 19 | Sorbitol | 48 | 82 | 8 | 130 |
| 24 | Starch | 31 | 46 | 7 | 77 |
| 25 | Sucrose | 30 | 67 | 17 | 107 |
| 26 | Glucose | 30 | 91 | 5 | 121 |
| 27 | Xylose | 70 | 43 | 4 | 113 |
| 28 | Arabinose | 74 | 44 | 5 | 118 |

### EXAMPLES 25 TO 30

These examples demonstrate the benefits of use of a pre-reduction step.

The method of Example 1 was repeated except that the reaction temperature was initially controlled below the level previously employed for the hydrogenolysis of sugars. The sorbitol was replaced with glucose.

The results are set out in Table 6. It is noted that pre-reduction of the glucose at both 150° C. and 200° C. improves the selectivity of the reaction such that it is greater than that observed for sorbitol (Example 1). This may be an indication that some hydrogenolysis also takes place at the lower temperature.

TABLE 6

| | | | | Ethylene | Propylene | | Total (Propylene |
|---|---|---|---|---|---|---|---|
| Ex | Temp1° C. | Temp2° C. | Temp3/° C. | glycol | glycol | Glycerol | glycol + Ethylene |
| No | (Time hrs) | (Time hrs) | (Time hrs) | (mol %) | (mol %) | (mol %) | glycol) (mol %) |
| 25 | | | 250 (6) | 30 | 91 | 5 | 121 |
| 26 | | 150 (2) | 250 (4) | 57 | 90 | 9 | 147 |
| 27 | | 150 (2) | 225 (4) | 45 | 80 | 22 | 125 |
| 28 | | 200 (2) | 250 (2) | 58 | 93 | 34 | 151 |
| 29 | | 200 (2) | 250 (4) | 48 | 94 | 15 | 144 |
| 30 | 150 (2) | 200 (2) | 250 (2) | 49 | 92 | 19 | 141 |

### EXAMPLES 31 TO 33

These examples further demonstrate the use of a pre-reduction step using N-methyl pyrrolidone as a solvent.

The method of Example 1 was repeated except that the sorbitol was replaced with glucose, the 20 g tetrahydrofuran was replaced with 50 g N-methyl pyrrolidone and a pre-reduction step was included.

The results are set out in Table 7. Pre-reduction of the glucose at 200° C. followed by hydrogenolysis at a higher temperature increases the selectivity towards desirable products. However, increasing the temperature above 260° C. appears to have a detrimental effect.

TABLE 7

| | | | Ethylene | Propylene | | Total (Propylene |
|---|---|---|---|---|---|---|
| E.g. | Temp1/° C. | Temp2/° C. | glycol | glycol | Glycerol | glycol + |
| No | (Time/hrs) | (Time/hrs) | (mol %) | (mol %) | (mol %) | Ethylene glycol) |
| 31 | | 250 (6) | 19 | 51 | 1 | 70 |
| 32 | 200(2) | 260 (4) | 63 | 98 | <1 | 162 |
| 33 | 200(2) | 270 (4) | 59 | 50 | 2 | 109 |

### EXAMPLES 34 TO 38

These examples further illustrate the usefulness of a ‘pre-reduction’ step in the hydrogenolysis of C₅ alditols.

The method of Example 1 was repeated except that the sorbitol was replaced by xylose or arabinose (C₅ sugars) and a ‘pre-reduction’ step was employed as outlined below. In Example 38 a mixture of xylose and glucose is used.

The results are set out in Table 8.

TABLE 8

| | | | | | | | Total (Propylene |
|---|---|---|---|---|---|---|---|
| E.g. | | Temp1/° C. | Temp2/° C. | Ethylene | Propylene | Glycerol | glycol + Ethylene |
| No | Sugar | (Time/hrs) | (Time/hrs) | glycol (mol %) | glycol (mol %) | (mol %) | glycol) (mol %) |
| 34 | Xylose | | 250 (6) | 70 | 43 | 4 | 113 |
| 35 | Arabinose | | 250 (6) | 74 | 44 | 5 | 118 |
| 36 | Xylose | 200 (2) | 250 (4) | 49 | 44 | 1 | 93 |
| 37 | Arabinose | 200 (2) | 250 (4) | 79 | 79 | 6 | 158 |
| 38 | Glucose + | 200 (2) | 250 (4) | 72 | 63 | 10 | 135 |
| | Xylose | | | | | | |

### EXAMPLES 39 TO 45

These examples further illustrate the hydrogenolysis of C₅ aldoses using a pre-reduction step and N-methyl pyrrolidone as solvent.

The method of Example 1 was repeated except that the tetrahydrofuran was replaced with 50 g of N-methylene pyrrolidone and the sorbitol with xylose.

The results are set out in Table 9. It is noted that in contrast to the results obtained for tetrahydrofuran (Examples 31 to 33), pre-reduction is effective for xylose in N-methyl pyrrolidone. The best results appear to occur with a two hour pre-reduction at 200° C.

TABLE 9

| | | | | | | Total |
|---|---|---|---|---|---|---|
| | Temp1/ | Temp2/ | Ethyl | Propyl | | (propylene |
| | ° C. | ° C. | ene | ene | | glycol + |
| Ex | (Time/ | (Time/ | glycol | glycol | Glycerol | ethylene |
| No | hrs) | hrs) | (mol %) | (mol %) | (mol %) | glycol) (mol %) |
| 39 | | 260 (6) | 50 | 38 | 2 | 88 |
| 40 | | 250 (6) | 45 | 47 | <1 | 92 |
| 41 | 200 (2) | 260 (4) | 79 | 76 | <1 | 155 |
| 42 | 200 (1) | 260 (5) | 40 | 76 | <1 | 116 |
| 43 | 200 (3) | 260 (4) | 79 | 39 | <1 | 118 |
| 44 | 200 (2) | 260 (2) | 77 | 74 | <1 | 151 |
| 45 | 200 (2) | 260 (6) | 75 | 56 | 1 | 131 |

### EXAMPLES 48 TO 49

These further demonstrate the suitability of the catalyst for the hydrogenolysis of a range of substrates.

The method of Example 1 was repeated except that the tetrahydrofuran was replaced by 50 g of N-methyl pyrollidone as the solvent, the sorbitol with a range of other substrates and a pre-reduction step was employed. The reaction therefore consisted of 2 hrs at 200° C. followed by 4 hrs at 250° C.

The results are set out in Table 10.

TABLE 10

| | | | | | Total |
|---|---|---|---|---|---|
| | | Ethylene | Propylene | | (propylene |
| Ex | | glycol | glycol | Glycerol | glycol + ethylene |
| No | Substrate | (mol %) | (mol %) | (mol %) | glycol) (mol %) |
| 46 | Glucose | 63 | 98 | <1 | 162 |
| 47 | Mannose | 72 | 81 | 8 | 153 |
| 48 | Mannitol | 77 | 82 | 2 | 159 |
| 49 | Ribose | 80 | 54 | 11 | 134 |

### EXAMPLES 50 TO 52

These examples explore the effect of the water concentration.

The method of Examples 39 to 45 was repeated except that glucose was employed as the substrate, and the amounts of water and glucose were modulated as set out in Table 11.

TABLE 11

| | | | | | | Total |
|---|---|---|---|---|---|---|
| | | | | | | (Propylene |
| | | | | | | glycol + |
| | | | Ethylene | Propylene | | Ethylene |
| E.g. | Water | Glucose | glycol | glycol | Glycerol | glycol) |
| No | (g) | (g) | (mol %) | (mol %) | (mol %) | (mol %) |
| 50 | 50 | 20 | 63 | 98 | <1 | 162 |
| 51 | 42 | 28 | 67 | 111 | 1 | 187 |
| 52 | 20 | 20 | 84 | 70 | 8 | 154 |

### EXAMPLES 53 TO 55

These examples explore the effect of added base and illustrate that the addition of base does not promote the selectivity of the catalyst as described in other patents. The method of Example 1 was repeated except that an amount of base was added to the reaction. In both cases this caused a small reduction in the amount of desirable products produced. The results are set out in Table 12.

TABLE 12

| | | | | | | Total |
|---|---|---|---|---|---|---|
| | | | | | | (Propylene |
| | | | | | | glycol + |
| | | | Ethylene | Propylene | | Ethylene |
| E.g | | | glycol | glycol | Glycerol | glycol) |
| No | Additive | Solvent | (mol %) | (mol %) | (mol %) | (mol %) |
| 53 | None | THF | 48 | 82 | 8 | 130 |
| 54 | NaOH | THF | 45 | 76 | 2 | 121 |
| 55 | NH4OH | THF | 42 | 36 | 1 | 78 |

### EXAMPLES 56 TO 59

These examples consider the effect of the reaction period and illustrates that the product profile may be varied by varying the reaction period and further illustrates the temperature range over which the catalyst is active.

The method of Example 1 was repeated except that the reaction temperature and reaction period were varied as described in Table 13.

TABLE 13

| | | | | | | Total | |
|---|---|---|---|---|---|---|---|
| | | | | | | (Propylene | |
| | | | Eth- | Propyl- | | glycol + | |
| | | | ylene | ene | | Ethylene | Conver- |
| Ex | Temp | Time | glycol | glycol | Glycerol | glycol) | sion |
| No | (° C.) | (hrs) | (mol %) | (mol %) | (mol %) | (mol %) | (wt %) |
| 56 | 250 | 6 | 48 | 82 | 8 | 130 | >99 |
| 57 | 250 | 3 | 44 | 76 | 16 | 120 | >99 |
| 58 | 200 | 6 | 46 | 40 | 45 | 86 | 72 |
| 59 | 150 | 20 | 9 | 9 | 10 | 18 | >2 |

### EXAMPLES 60 TO 63

These examples demonstrate that with a less volatile solvent the catalyst is relatively insensitive to pressure.

The method of Examples 39 to 45 were repeated except that the reaction pressure was varied. Where sorbitol was employed as a substrate, no ‘pre-reduction’ step was involved and the total reaction period was 6 hrs. The results are set out in Table 14.

TABLE 14

| | | | | | | Total |
|---|---|---|---|---|---|---|
| | | | | | | (Propylene |
| | | | | | | glycol + |
| | | | Ethylene | Propylene | | Ethylene |
| E.g | Pressure | | glycol | glycol | Glycerol | glycol) |
| No | (psig) | Substrate | (mol %) | (mol %) | (mol %) | (mol %) |
| 60 | 1180 | Sorbitol | 74 | 80 | 3 | 154 |
| 61 | 1000 | Sorbitol | 56 | 67 | 5 | 123 |
| 62 | 1213 | Glucose | 69 | 81 | 10 | 150 |
| 63 | 1000 | Glucose | 84 | 70 | 8 | 154 |

### EXAMPLES 64 TO 71

These examples illustrate that certain additives can increase the selectivity to the desired product.

The method of Example 1 was repeated, except that an amount of triphenylphosphine was added to the reaction. Where N-methyl pyrrolidone was employed as a solvent, 50 g of N-methyl pyrrolidone were used instead of 20 g of tetrahydrofuran The results are set out in Table 15. It can be seen that TPP has a beneficial effect in the presence of certain solvents, notably NMP.

TABLE 15

| | | | | | | | Total |
|---|---|---|---|---|---|---|---|
| | | | | | | | (Propylene |
| | | | | | | | glycol + |
| | | | | Ethyl- | Propyl- | Glycer- | Ethylene |
| E.g. | Addi- | | Pressure | ene glycol | ene glycol | ol | glycol) |
| No | tive | Solvent | (psig) | (mol %) | (mol %) | (mol %) | (mol %) |
| 64 | None | THF | 1000 | 48 | 82 | 8 | 130 |
| 65 | TPP | THF | 1000 | 58 | 72 | 1 | 130 |
| 66 | TPP | THF | 1000 | 51 | 78 | 1 | 129 |
| 67 | TPP | THF | 1000 | 51 | 80 | 2 | 131 |
| 68 | TPP | THF | 1265 | 56 | 67 | 16 | 123 |
| 69 | TPP | NMP | 1000 | 76 | 76 | 3 | 152 |
| 70 | None | NMP | 1000 | 56 | 67 | <1 | 123 |
| 71 | TPP | NMP | 1242 | 68 | 73 | 4 | 141 |

### EXAMPLES 72 TO 82

These examples consider the effect of changing the phosphine and illustrates that tridentate phosphines, in particular facially co-ordinating tripodal phosphines are particularly useful for this reaction. This also provides a comparison with TPP which was employed in the prior art as the ligand of choice.

The method of Example 1 was repeated except that the triphos was replaced by an amount of another ligand as indicated in Table 16.

TABLE 16

| | | | | | | Total |
|---|---|---|---|---|---|---|
| | | | | | | (Propylene |
| | | | | | | glycol + |
| | | Ligand/ | Ethylene | Propylene | | Ethylene |
| Ex | Ligands | Ru | glycol | glycol | Glycerol | glycol) |
| No | (s) | ratio | (mol %) | (mol %) | (mol %) | (mol %) |
| 72 | Triphos | 1.2 | 48 | 82 | 8 | 130 |
| 73 | Triphos/ | 1.2 | 51 | 80 | 2 | 131 |
| | TPP | | | | | |
| 74 | Dppe | 2.5 | 10 | 9 | <1 | 19 |
| 75 | Dppp | 2.6 | 29 | 30 | 1 | 59 |
| 76 | TPP | 4 | 4 | 0.1 | 2 | 4 |
| 77 | None | — | 8 | 2 | 1 | 10 |
| 78 | Dppp | 2.6 | 33 | 35 | 9 | 68 |
| 79 | Dppp | 1.3 | 8 | 3 | 1 | 11 |
| 80 | Dppp | 4.6 | 25 | 27 | 8 | 52 |
| 81 | PCy3 | 7.8 | 2 | 0 | 0 | 2 |
| 82 | ‘Normal’ | 1.2 | 24 | 35 | 29 | 59 |
| | Triphos | | | | | |

“Dppe” is 1,2-bis(diphenylphosphino)ethane, “Dppp” is 1,3-bis(diphenylphosphino)propane, “normal” triphos is 1,1-bis (diphenylphosphinoethyl)phenylphosphine.

### EXAMPLES 83 AND 84

A second set of tests were performed using 50 g of N-methyl pyrrolidone as a solvent, and at a water loading of 50 g. For the Ethphos ligand, the catalyst was pre-formed by heating the ruthenium and phosphine to 200° C. for 1 hr in the absence of water in N-methyl pyrrolidone. The results are set out in Table 17

TABLE 17

| | | | | | | Total |
|---|---|---|---|---|---|---|
| | | | | | | (Propylene |
| | | | | | | glycol + |
| | | | Ethylene | Propylene | | Ethylene |
| Ex | Ligands | Ligand/ | glycol | glycol | Glycerol | glycol) |
| No | (s) | Ru ratio | (mol %) | (mol %) | (mol %) | (mol %) |
| 83 | Triphos | 1.2 | 48 | 82 | 8 | 130 |
| 84 | Ethphos | 1.0 | 71 | 54 | 19 | 125 |

Ethphos is 1,1,1-tris(diethylphosphinomethyl)ethane.

### EXAMPLE 85

This illustrates that polymeric aldoses such as cellulose will undergo hydrogenolysis in the presence of the catalyst. 11.3 g of an NMP solution containing 0.18 g of Ru(ac ac)3 and 0.38 g of triphos (which had been heated to 200° C. under nitrogen to coordinate the triphos to the ruthenium), 70 g of water and 20 g of cellulose (ex Aldrich,20 micron powder) were loaded into a 300 ml hastelloy autoclave. The autoclave was sealed, purged with hydrogen, pressurised to 500 psig with Hydrogen and then heated to 200C with stirring. Once 200° C. was attained the pressure was increased to 900psig and the reaction started. After 2 hrs the reactor was heated to 250° C. and the pressure increased to 1000 psig. The reaction was left for a further four hours under regulator control. At the end of the reaction 98.3 g of product were recovered containing an orange solution and a solid material (6.1 g, unreacted cellulose). The product was analysed by GC using an internal standard. Mol % selectivities EG (52) PG (44). Other products identified in the product mixture by GC-MS include 1-propanol, ethanol, 1-butanol, 1-pentanol, 2-pentanol, 1,2-butanediol and 1,2-petanediol.

1. A process for the hydrogenolysis of a sugar feedstock in the presence of a catalyst comprising:
(a) ruthenium or osmium; and
(b) an organic phosphine;
and wherein the hydrogenolysis is carried out in the presence of water and at a temperature of greater than 150° C.

2. A process according to claim 1 wherein the sugar feedstock is a feedstock comprising one or more of polyols, alditols, aldoses and polymers of aldoses.

3. A process according to claim 2 wherein the polymers of aldoses are starch or cellulose.

4. A process according to claim 2 wherein the alditols and aldoses suitable for use in the process of the present invention are those being from C₃ to C₁₂.

5. A process according to claim 4 wherein the alditols and aldoses suitable for use in the process of the present invention are those being from C₃ to C₆.

6. A process according to claim 1 wherein the feedstock is selected from glucose, sucrose, xylose, arabinose and mannose.

7. A process according to claim 1 wherein water is present as the solvent for the reaction.

8. A process according to claim 1 wherein the sugar feedstock or the product of the reaction is the solvent and water is added as an additive in the solvent.

9. A process according to claim 1 wherein a solvent is used and water is added as an additive in the solvent.

10. A process according to claim 9 wherein suitable solvents are selected from tetraethyleneglycol dimethyl ether, tetrahydrofuran, amides, lactams, N-methyl caprolactam, N-methyl pyrrolidone, diethyl ether, ethyleneglycol dimethylether, dioxane, 2-propanol, 2-butanol, secondary alcohols and tertiary alcohols.

11. A process according to claim 1 wherein the ruthenium is provided as a ruthenium compound.

12. A process according to claim 11 wherein the ruthenium compound is a nitrate, sulphate, carboxylate, beta diketone, and carbonyls.

13. A process according to claim 1 wherein the ruthenium is present in an amount of from 0.0001 to 5 mol as ruthenium per liter of reaction solution.

14. A process according to claim 1 wherein the phosphine is selected from mono, bi and tridentate phosphines.

15. A process according to claim 1 wherein the phosphine is selected from trialkylphosphines, dialkylphosphines,monoalkylphosphines, triarylphosphines, diarylphosphine, monoarylphosphines, diarylmonoalkyl phosphines anddialkylmonoaryl phosphines.

16. A process according to claim 15 wherein the phosphine is selected from tris-1,1,1-(diphenylphosphinomethyl)methane, tris-1,1,1-(diphenylphosphinomethyl)ethane, tris-1,1,1-(diphenylphosphinomethyl)propane, tris-1,1,1-(diphenylphosphino-methyl)butane, tris-1,1,1-(diphenylphosphinomethyl)2,2dimethylpropane, tris-1,3,5-(diphenyl-phosphino-methyl)cyclohexane, tris-1,1,1-(dicyclohexylphosphinomethyl)ethane, tris-1,1,1-(dimethylphosphinomethyl)ethane, tris-1,1,1-(diethylphosphinomethyl)ethane, 1,5,9-triethyl-1,5-9-triphosphacyclododecane, 1,5,9-triphenyl-1,5-9-triphosphacyclododecane, bis(2-diphylephosphinoethyl)phenylphosphine, bis-1,2-(diphenylphosphino)ethane, bis-1,3-(diphenylphosphino)propane, bis-1,4-(diphenylphosphino)butane, bis-1,2-(dimethyl phosphino)ethane, bis-1,3-(diethylphosphino)propane,bis-1,4-(dicyclohexylphosphino)butane, tricyclohexylphosphine, trioctylphosphine, trimethylphosphine, tripyridylphosphine and triphenylphosphine

17. A process according to claim 13 wherein the phosphine is a tridentate phosphine.

18. A process according to claim 17 wherein-the tridentate phosphine is tris-1,1,1 -(diarylphosphinomethylalkane or tris-1,1,1-(dialkylphosphinomethyl) alkane.

19. A process according to claim 1 wherein the phosphine compound is present in an amount of from 0.0001 to 5 mol as phosphine per liter of reaction solution.

20. A process according to claim 1 wherein a base is added.

21. A process according to claim 20 wherein the base is an amine.

22. A process according to claim 1 wherein a second phosphine is added to increase the selectivity.

23. A process according to claim 22 wherein the second phosphine is one being more weakly coordinating than the phosphine.

24. A process according to claim 1 wherein the temperature is from about 190° C. to about 260° C.

25. A process according to claim 1 wherein the reaction pressure is from about 250 psig to about 2000 psig.

26. A process according to claim 1 wherein the sugar feedstock is an aldose and a pre-reduction step is included.

27. A process according to claim 22 wherein the temperature of the pre-reduction step is from about 150° C. to about 250° C.

28. A process according to claim 26 wherein the pressure of the pre-reduction step is from about 600 to about 1000 psig.

29. A process according to claim 1 wherein the catalyst is regenerated in the presence of the water and hydrogen.
